(19) United States
(12) Patent Application Publication
(10) Pub. No.: US 2025/0064891 A1
(43) Pub. Date: Feb. 27, 2025

(54) HOST PROTEASES ESSENTIAL FOR PARAINFLUENZA SPREAD IN THE HUMAN LUNG: POTENTIAL TARGETS FOR ANTIVIRAL INTERVENTIONS

(71) Applicant: The Trustees of Columbia University in the City of New York, New York, NY (US)

(72) Inventors: Anne MOSCONA, New York, NY (US); Kyle STEARNS, New York, NY (US); Matteo POROTTO, New York, NY (US)

(21) Appl. No.: 18/946,680

(22) Filed: Nov. 13, 2024

Related U.S. Application Data
(63) Continuation of application No. PCT/US2023/066930, filed on May 12, 2023.
(60) Provisional application No. 63/341,956, filed on May 13, 2022.

(51) Int. Cl. A61K 38/17; A61K 31/132; A61K 31/245; A61P 31/16

(52) U.S. Cl. CPC A61K 38/1709; A61K 31/132; A61K 31/245; A61P 31/16

(57) ABSTRACT

Transmembrane proteases are necessary for HPIV3 fusion protein cleavage in the human lung. HPIV3 fusion protein cleavage in the human lung is not furin dependent as previously thought, but instead is facilitated by transmembrane proteases, typically targeting TMPRSS2 expression. Serine protease inhibitors may be used in this manner to treat HPIV in a patient. Particular serine protease inhibitors shown to be effective include nafamostat, camostat, aprotinin, and leupeptin. Method of treating HPIV use serine protease inhibitors as treating agents.

This application is a Continuation of International Application No. PCT/US2023/066930, filed on May 12, 2023, claims priority to U.S. Provisional Application No. 63/341,956 filed on May 13, 2022, each of which are herein incorporated by reference in its entirety.

All patents, patent applications and publications cited herein are hereby incorporated by reference in their entirety. The disclosures of these materials in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT SUPPORT

This invention was made with government support under grant DK007328, awarded by the National Institutes of Health. The government has certain rights in the invention.

### BACKGROUND

Entry and infection by human parainfluenza virus 3 (HPIV3) into a target cell is dependent on highly specialized viral fusion machinery that has been honed by evolution to have a balance of receptor binding affinity, membrane fusion activation, and neuraminidase driven release of virus, that favor spread within and between hosts. The regulatory contributions of host factors necessary for the activation of viral proproteins remain undefined yet are key for host and tissue tropism and pathogenesis.

HPIV3 infection is driven by the coordinated action of viral surface glycoproteins hemagglutinin-neuraminidase (HN) and fusion protein (F). Upon HN binding to sialic acid bearing proteins on the target cell, HN triggers F to insert into the target cell membrane and drive virion-cell membrane fusion. For infection to occur, the fusion protein precursor (F0) must first be cleaved by host proteases into its active form, composed of the disulfide-linked subunits F1 and F2. F0 cleavage has been thought to be executed during viral glycoprotein transit through the trans-Golgi network by the ubiquitously expressed furin, because F0 contains a dibasic cleavage site (R-T-K-R).

### BRIEF SUMMARY

We found that the dibasic cleavage site underlying the prior assumption is an artifact of laboratory adaptation, whereas circulating strains of HPIV3 actually have a monobasic cleavage site (R-T-E-R) that is cleaved by a protease not yet identified. As a consequence, furin-cleaved laboratory adapted strains promiscuously infect cell types, but authentic clinical viruses exhibit tight tropism for human lung.

To study viral tropism, we sought to identify the protease responsible for authentic F0 cleavage in vivo. We assessed protein expression in tissues and cell culture models that support the growth of authentic HPIV3 to identify candidate proteases that may process HPIV3 F in vivo.

The resulting data indicates F0 protein cleavage occurs at the cell surface facilitated by transmembrane proteases, in distinction to the previous notion of F0 processing by furin in the trans-Golgi network. These findings support an alternative mechanism of F activation in vivo, reliant on host factors expressed in a narrower subset of cells. Understanding how F is processed in the human lung is important for understanding viral tropism and host factors involved in regulating infection.

In distinction to the previous notion of F0 processing by intracellular furin in the trans-Golgi network, furin is not sufficient for the spread of clinical strains of HPIV3.
Gain-of-function experiments revealed candidate proteases that are sufficient to cleave F0 and produce infectious virions. For example, extracellular TMPRSS2 is sufficient to cleave F0 and produce infectious virions.
Extracellular serine protease inhibitors, aprotinin and leupeptin, blocked HPIV3 F cleavage and inhibited viral spread.
Nafamostat (a potent serine protease inhibitor) treatment reduced HPIV3 spread in nasal tissue of cotton rats.

These results show that HPIV3 F is cleaved by extracellular serine proteases, distinct from the intracellular, furin-dependent cleavage previously thought to occur. Our recent work applies this observation for the novel application of inhibitors, including serine protease inhibitors among other potential host factors, for the treatment of HPIV3. These findings support an alternative mechanism of F activation in vivo and a novel strategy for regulating HPIV3 spread in the human lung.

### BRIEF DESCRIPTION OF FIGURES

FIG. 1: Human parainfluenza viruses in circulation have different fusion protein cleavage motifs than lab adapted strains. Patient viral isolates were directly sequenced (without passage in cell culture) and analyzed using metagenomic next-generation sequencing (mNGS) to assess genome-wide mutational changes. Listed are cleavage motif sequence alignments of laboratory adapted and patient derived HPIV3. Reported are the observed sequence frequencies and the amino acid sequences four amino acids upstream and downstream of the HPIV F1/F2 cleavage site.

FIG. 2A-FIG. 2B: TMPRSS2 expression is sufficient for HPIV3 F cleavage in HEK293T cells and necessary for infectious viral particle production by Calu-3 cells. 293T cells were transfected with a TMPRSS2 open reading frame (ORF), TMPRSS2 CRISPR activating (CRISPRa), or a non-specific guide RNA CRISPRa control plasmid. (A) TMPRSS2 mRNA expression relative to non-transfected 293T cells determined by qRT-PCR. (B) HPIV-3 F E108 (left) or HPIV-3 F K108 (right) titer in 293T-TMPRSS2 ORF, 293T-TMPRSS2 CRISPRa, and non-transfected cell culture media. Values are means and SEM from three biological replicates.

FIG. 3A-FIG. 3B: Cell impermeable serine protease inhibitors, aprotinin and leupeptin, block HPIV3 F cleavage and reduces production of infectious viral particles. (A) Calu-3 cells and (B) HAE were inoculated with HPIV-3 F E108 (left) or HPIV-3 F K108 (right). After inoculation, cells were treated with the indicated concentrations of aprotinin or leupeptin. HPIV3 infected Calu-3 cell culture media and HAE apical wash resolved by reducing SDS-PAGE and immunoblotted with anti-HPIV3 F antibody. HPIV3 titer in cell culture media and HAE apical washes 3 days after infection. Values are means and SEM from 1-3 biological replicates.

FIG. 4: Serine protease inhibitors block HPIV3 F cleavage and reduces production of infectious viral particles. Calu-3 cells were inoculated with HPIV-3 F E108 (red). After inoculation, cells were treated with the indicated concentrations of protease inhibitor. HPIV3 titer in cell culture media shows significant reduction in HPIV3 titer in serine protease inhibitor treated wells. Values are means and SEM from one biological replicate.

FIG. 5: Nafamostat blocks HPIV3 spread in vivo. Groups of 5 cotton rats were intranasally infected with CI HPIV-3 F E108 (left) or HPIV-3 F K108 (right) and then administered nafamostat at 3 mg/kg body weight. Intranasal nafamostat treatments were continued daily, and the cotton rats were sacrificed 3 days after infection. Viral titer (LogTCID50 per gram of nasal tissue) was determined by plaque assay. Values are means and SEM.

FIG. 6: Steps of HPIV3 entry. A1) Virion comes in close proximity to host cell. A2) HPIV3 HN binds to host cell sialic acid bearing protein. A3) Sialic acid bound HN induces F to fold into its elongated state, inserting its hydrophobic residues into the host membrane. A4) F mediates membrane fusion and enters its post-fusion conformation.

FIG. 7A-FIG. 7B: Screening for cell models capable of cleaving HPIV3 F E108. A) HPIV3 F E108 or HPIV3 F K108 infected cell media were run on a gel and blotted for HPIV3 F protein. B) Infected cell media collected 1-3 days post infection was titered in Vero cells. Calu-3, RHMK, and MDBK cells efficiently cleave F E108and produce infectious viral particles.

FIG. 8A-FIG. 8B: Cell impermeable serine protease inhibitors block HPIV3 F cleavage and reduce production of infectious viral particles. A) Calu-3 cells infected with HPIV3 F E108 (left) or HPIV3 F K108 (right) were treated with serine protease inhibitors leupeptin or aprotinin. Virions released by the infected cells into the media were titered. B) Western blotting for HPIV3 F in Calu-3 cell media. Leupeptin and aprotinin treated cells have reduced viral titer and reduced HPIV3 F E108 cleavage.

FIG. 9: F0 cleavage at the site of viral fusion promotes more robust infection. HPIV3 F E108 bearing F0 and HPIV3 F K108 were either preincubated with TPCK treated trypsin prior to target cell binding or bound to target cells at 4° C. and then incubated with TPCK treated trypsin. Cleavage of F0 on target cell bound virions increased infection efficiency.

### DETAILED DESCRIPTION

Entry by human parainfluenza virus 3 (HPIV3) into a target cell is dependent on highly specialized viral fusion machinery that has been honed by evolution to have a balance of receptor binding affinity, membrane fusion activation, and neuraminidase driven release of virus, that favor spread within and between hosts. The regulatory contributions of host factors necessary for the activation of viral proproteins remain undefined yet are key for host and tissue tropism and pathogenesis.

HPIV3 infection is driven by the coordinated action of viral surface glycoproteins hemagglutinin-neuraminidase (HN) and fusion protein (F). Upon HN binding to sialic acid bearing proteins on the target cell, HN triggers F to insert into the target cell membrane and drive virion-cell membrane fusion.

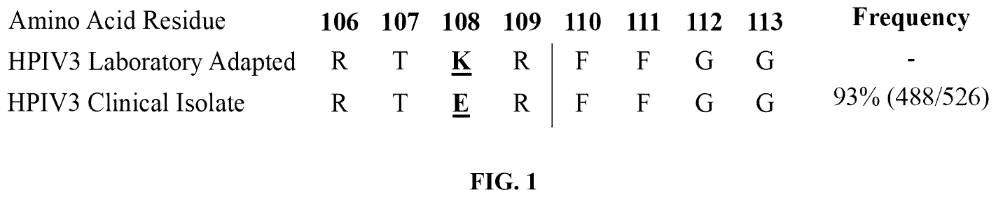

For infection to occur, the fusion protein precursor (F0) must first be cleaved by host proteases into its active form, composed of the disulfide-linked subunits F1 and F2. F0 cleavage has been thought to be executed during viral glycoprotein transit through the trans-Golgi network by the ubiquitously expressed furin, because F0 contains a dibasic cleavage site (R-T-K-R) (FIG. 1).

However, we found that the dibasic cleavage site underlying the assumption is an artifact of laboratory adaptation, whereas circulating strains of HPIV3 instead have a monobasic cleavage site (R-T-E-R) that is cut by an unidentified protease. Therefore, furin-cleaved laboratory adapted strains promiscuously infect hosts and cell types, but authentic clinical viruses exhibit tight tropism for human lung.

To study viral tropism, we sought to identify the protease responsible for authentic F0 cleavage in vivo. We assessed protein expression in tissues and cell culture models that support the growth of authentic HPIV3 to identify candidate proteases that may process HPIV3 F in vivo.

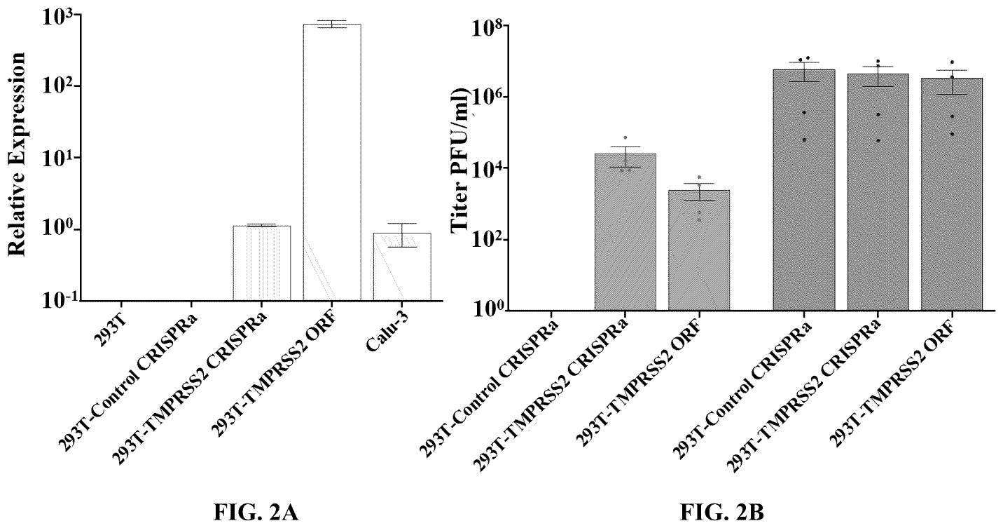

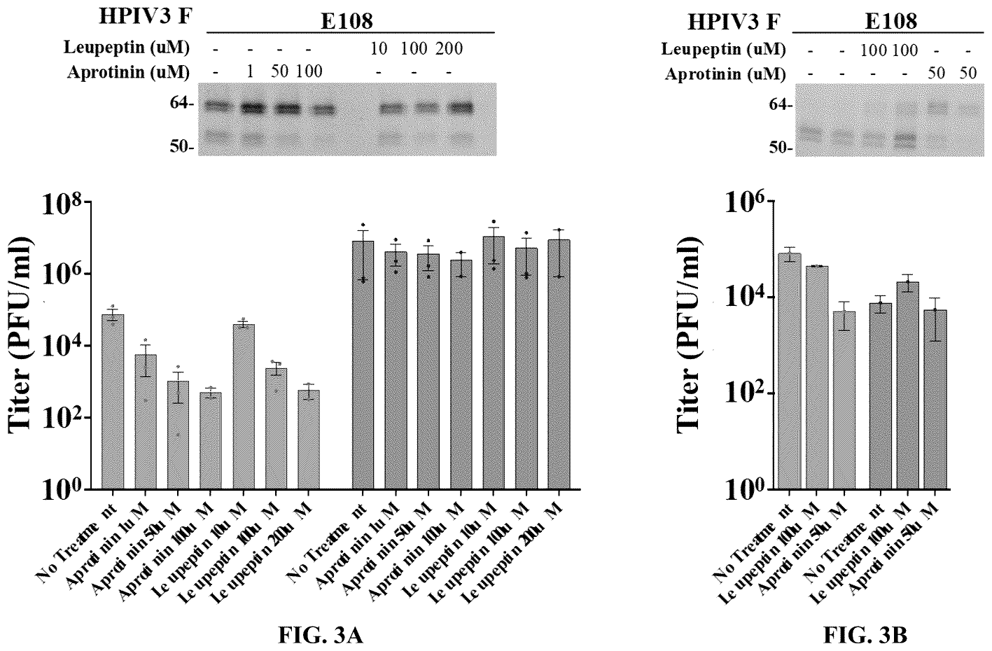

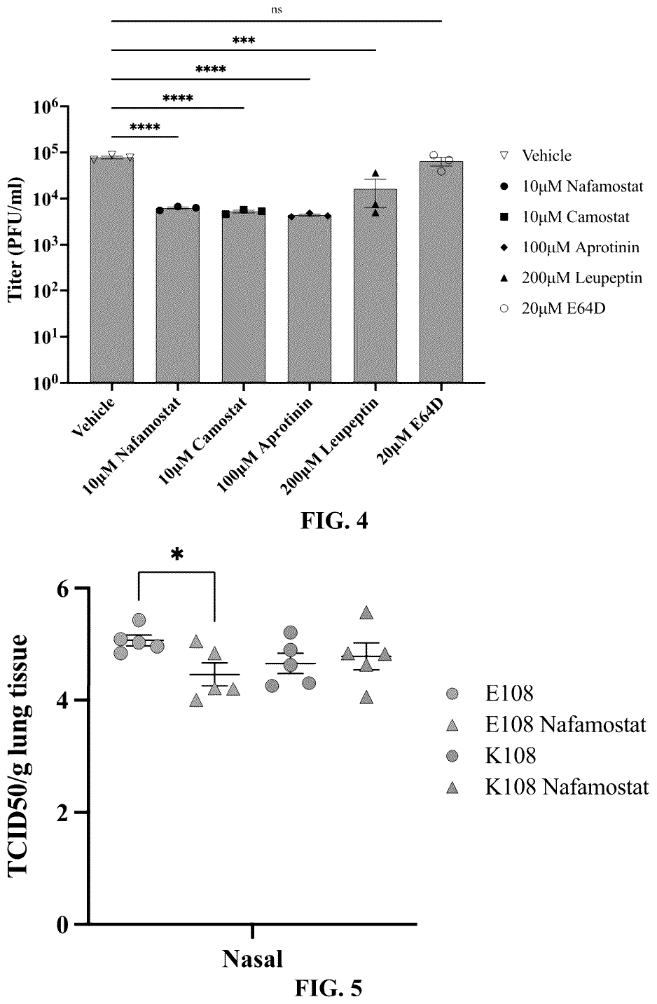

In distinction to the previous notion of F0 processing by intracellular furin in the trans-Golgi network, furin is not sufficient for the spread of clinical strains of HPIV3.
Gain-of-function experiments revealed that extracellular TMPRSS2 is sufficient to cleave F0 and produce infectious virions (FIG. 2).
Extracellular serine protease inhibitors, aprotinin and leupeptin, blocked HPIV3 F cleavage and inhibited viral spread (FIG. 3-4).
Nafamostat (potent serine protease inhibitor) treatment reduced HPIV3 spread in nasal tissue of cotton rats (FIG. 5).

These results show that HPIV3 F is cleaved extracellularly, distinct from intracellular cleavage previously thought. Our work applies this observation for the novel application of inhibitors, including serine protease inhibitors among other potential host factor, for the treatment of HPIV3. These findings demonstrate an alternative mechanism of F activation in vivo, and thus a novel strategy for regulating HPIV3 spread in the human lung. For example, inhibiting serine proteases, such as TMPRSS2, blocks HPIV3 F cleavage, inhibiting or preventing viral spread or infection.

Serine protease inhibitors demonstrate extracellular activity, or both intracellular and extracellular activity. Protease inhibitors shown here to be effective inhibitors of serine proteases, such as TMPRSS2, include aprotinin and leupeptin, which exhibit extracellular activity, as well as nafamostat and camostat, which exhibit both intracellular and extracellular activity. Other serine protease inhibitor drugs expected to be useful with this subject matter include alirocumab, alpha-1-proteinase inhibitor, apixaban, argatroban, benzamidine, berotralstat, betrixaban, bivalirudin, CGS-27023, cholesterol sulfate, chymostatin, conestat alfa, dabigatran, dabigatran etexilate, darexaban, desirudin, edoxaban, evolocumab, fondaparinux, gabexate, GW-813893, human C1-esterase inhibitor, idraparinux, lepirudin, letaxaban, melagatran, otamixaban, rivaroxaban, rosmarinic acid, sivelestat, ulinastatin, and ximelagatran.

Some embodiments of the instant subject matter provide compositions useful for treating or preventing HPIV3, the compositions comprising at least one component effective for blocking HPIV3 F cleavage, and viral spread. In some embodiments, the at least one component inhibits TMPRSS2, thereby blocking cleavage of F0. In certain embodiments, the at least one component is at least one serine protease inhibitor.

In certain embodiments, the at least one serine protease inhibitor includes at least one of the following: aprotinin, leupeptin, nafamostat, and camostat. In other embodiments, the protease inhibitor includes at least one of the following: aprotinin, leupeptin, nafamostat, camostat, alirocumab, alpha-1-proteinase inhibitor, apixaban, argatroban, benzamidine, berotralstat, betrixaban, bivalirudin, CGS-27023, cholesterol sulfate, chymostatin, conestat alfa, dabigatran, dabigatran etexilate, darexaban, desirudin, edoxaban, evolocumab, fondaparinux, gabexate, GW-813893, human C1-esterase inhibitor, idraparinux, lepirudin, letaxaban, melagatran, otamixaban, rivaroxaban, rosmarinic acid, sivelestat, ulinastatin, and ximelagatran. In some embodiments, the at least one serine protease inhibitor includes at least one of the following: aprotinin, camostat, leupeptin, and nafamostat.

In some embodiments, the at least one serine protease inhibitor includes nafamostat. In other embodiments, the at least one serine protease inhibitor includes at least one of aprotinin and leupeptin.

Other embodiments provide pharmaceutical compositions including any of the compositions listed above.

Other embodiments provide methods of treating or preventing a HPIV3 infection by administering any of the compositions listed above.

Yet other embodiments provide use of any of the compositions listed here in preparing a pharmaceutical composition for treating or preventing a HPIV3 infection.

### Specific Aims

HPIV host factor dependence and evasion in the human lung: Our goals are (1) to identify host proteases required for human parainfluenza virus 3 (HPIV3) fusion protein activation as well as the consequences of their selective expression on viral tropism, and (2) to understand the mechanisms by which viral dependence on these proteases supports viral propagation in the human lung. The results of these goals will elucidate essential host factors for HPIV3 spread and novel mechanisms by which the virus exploits these host factors to selectively target cells and proliferate in the human respiratory system.

Background: HPIVs are negative-sense single-strand RNA viruses with envelopes coated in two surface glycoproteins, hemagglutinin-neuraminidase (HN), and fusion protein (F). The coordinated actions of these two glycoproteins facilitate fusion of the virus and host membranes. The fusion cascade is initiated by HN binding to sialic acid-bearing receptors on the surface of the target cell. Bound HN then triggers the insertion of the F protein into the cell membrane. Refolding of the F protein into its post-fusion state drives the fusion of the host membrane and virus, and the subsequent release of viral contents into the host cell.

For infection to occur, the F protein precursor (F0) must first be cleaved by host proteases into its active form, composed of the disulfide-linked subunits F1 and F2. This cleavage event was thought to be achieved intracellularly by the ubiquitously expressed protease furin, because F0 contains a dibasic cleavage site (R-T-K-R). See FIG. 1. However, our laboratory demonstrated that the dibasic cleavage site underlying the assumption is an artifact of laboratory adaptation, and that circulating strains of HPIV3 instead have a monobasic cleavage site (R-T-E-R) that is cleaved by an unidentified protease. Therefore, furin-cleaved laboratory adapted strains promiscuously infect hosts and cell types, but authentic clinical viruses exhibit tight tropism for human lung. To study viral tropism, we sought to identify the protease responsible for authentic F0 cleavage in vivo. We assessed protein expression in tissues and cell culture models that support the growth of authentic HPIV3 to identify candidate proteases that may process HPIV3 F in vivo.

In distinction to the previous notion of F0 processing by intracellular furin in the trans-Golgi network, furin is not sufficient for the spread of clinical strains of HPIV3.

As a consequence, the cellular location of F0 cleavage and the biological implications of this critical activation step in the virus life cycle remain unknown.

Consequences of a monobasic cleavage motif in circulating strains of HPIV3: Relative to promiscuous furin-dependent viruses capable of being released as infectious virions by nearly all cell types, circulating HPIV3 can only be activated by certain cell types that express the proteases necessary for cleaving F0 bearing a monobasic cleavage motif. Our laboratory has demonstrated that authentic lung models, including human airway epithelium and lung organoid, can propagate clinical isolates of HPIV3 without exerting selective pressure on the viruses and giving rise to HPIV3 with lab adapted features. Conservation of the F protein monobasic cleavage motif in circulating strains and authentic lung models suggests the motif is important for viral fitness in the human lung. At this time, the biological mechanisms through which this fitness is conferred have not been explored. In aim 1, we will identify host proteases that are sufficient for circulating HPIV3 F0 cleavage and assess the impact of their expression on viral tropism in authentic lung models. In aim 2, we will ascertain if clinical isolate F confers a sufficient advantage to become dominant during coinfection with lab adapted virus in authentic lung models, and explore mechanisms by which furin-independent F cleavage may support viral spread in the human lung.
Aim 1: What host factors are required for circulating HPIV3 fusion protein cleavage and successful viral entry? We hypothesized that transmembrane proteases are necessary for HPIV3 fusion protein cleavage in the human lung. We identify proteases sufficient for HPIV3 F activation with gain-of-function and loss-of-function experiments. Cells lacking the protease activity to cleave F0 will be transfected with CRISPRa and open reading frame plasmids for candidate proteases, and then infected with circulating HPIV3. Viral spread and HPIV3 F protein cleavage will be monitored to identify proteases that confer the gain-of-functions sufficient for viral propagation. Complementary loss-of-function experiments will explore the necessity of the proteases for HPIV3 F propagation in the authentic lung models. Next, we will determine the phases of the virus infection cycle in which F cleavage may occur.

A series of in vitro assays will be performed to assess whether the proteases are able to cleave F0 upon HN-receptor engagement, or if F0 is primarily cleaved during egress. Finally, we will assess the impact of protease expression on viral tropism by employing a combination of immunohistochemistry and flow cytometry to spatially and quantitatively assess the relationship between protease expression and HPIV3 infection. These experiments will identify the host factors that are required for viral entry, characterize their mechanisms of F0 cleavage, and assess their impact on viral spread in the human lung.
Aim 2: Through what mechanisms does F cleavage by host proteases confer increased fitness in the human lung? We hypothesize that F proteins bearing the clinical isolate cleavage motif are selectively activated at advantageous times and proximities to target cells, facilitated by dependence on narrowly expressed host proteases. To assess the selective advantage of the circulating strain cleavage motif at the multicellular level, authentic lung models will be coinfected with varying proportions of HPIV3 bearing F with the clinical isolate or lab adapted cleavage motifs, and strain dominance will be monitored over time. Next, we will analyze the impact of cell surface cleavage of F0 on viral fitness by comparing the fusion efficiency of infections mediated by F cleaved before and after HN-receptor engagement. Finally, we will determine if the circulating strain cleavage site enables virions to limit untimely triggering of F0 and preserve viable fusion proteins until retained F0 proteins can be cleaved and activated by proteases at the surface of target cells. Taken together, the results of this aim will highlight if the clinical isolate F cleavage motif confers a competitive advantage that is evident in authentic lung models, and will explore novel functions of F0 for evading virucidal host factors and facilitating viral fusion upon reaching a target cell.
   A. Background, Significance and Preliminary Data The purpose of this endeavor is to support the investigation of paramyxovirus fusion machinery critical for viral entry into host cells. The goal is to identify host factors required for the activation of paramyxovirus fusion complex components, and to elucidate the evolutionary pressures favoring cell-specific proteolytic requirements in vivo. The results of the experiments outlined will identify host factors that are necessary for viral spread in the human lung and elucidate their role in determining viral tropism.
A.1. Human Parainfluenza Virus.

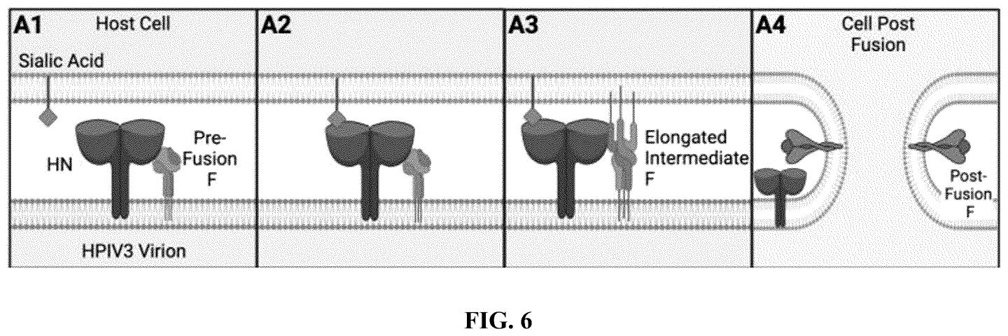

Human parainfluenza virus (HPIV) infections cause approximately 725,000 hospitalizations and more than 30,000 deaths in children under the age of 5 globally each year (2, 3). HPIVs, human respiratory syncytial virus (HRSV), and human metapneumovirus (HMPV) are major causes of lower respiratory infections and are responsible for the majority of cases of croup, bronchiolitis, and pneumonia in infants and children (3, 4). In the absence of targeted therapies or vaccines, corticosteroids have decreased hospitalizations for HPIV1-associated croup, but HPIV2 and HPIV3 infections remain untreatable (4-7).
A.2. HPIV3 Viral Fusion Machinery
   HPIV3 entry is mediated by the interaction of viral glycoproteins, hemagglutinin neuraminidase (HN) and fusion protein (F), with host-cell membranes. HPIV3 HN has the dual functions of hemagglutinin (binding sialic acid bearing proteins) and neuraminidase (facilitating release from the host via sialic acid cleavage) (8, 9). HPIV3 F is synthesized as a precursor (F0) that is cleaved to yield the pre-fusion F complex consisting of a trimer of C-terminal F1 subunits covalently linked via disulfide bonds with their respective N-terminal F2 subunits. Prior to engagement of a sialic acid-containing receptor, HN stabilizes the pre-fusion F. The trimeric F structure is maintained in metastable conformation that that can be activated by receptor-bound HN (10-15). Upon sialic acid engagement of the HN receptor, HN can either cleave sialic acid, thereby freeing the virion, or activate F into a transient intermediate state. Upon activation, the elongated transient intermediate state of the F protein extends and inserts its hydrophobic domain into the target cell. Refolding of the F protein into its post-fusion conformation mediates fusion of the viral and cell membranes, and allows the release of the viral genome into the target cell (FIG. 6) (5, 11, 12, 14-21).
   Viral fusion protein cleavage, a determinant of viral tropism: Paramyxovirus fusion proteins require cleavage by host proteases to be activated. Many paramyxoviruses, including HPIV3, have been reported to contain fusion proteins bearing a dibasic cleavage motif (R-X-K/R-R) that can be cleaved intracellularly by furin, which is ubiquitously expressed across cell types (22-24). As a result, fusion protein activation is not constrained by cell-specific protease expression and is dictated by other features of the host environment like the availability of target receptors.

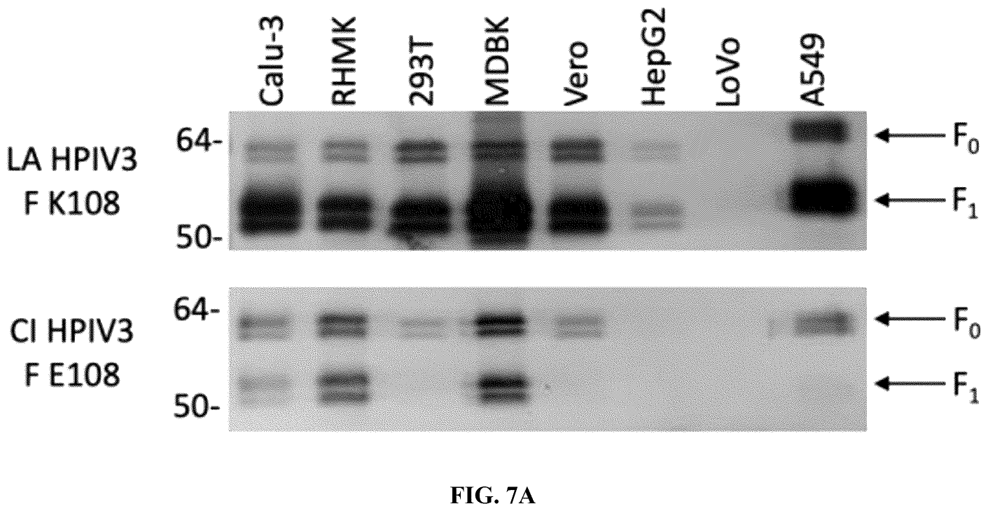

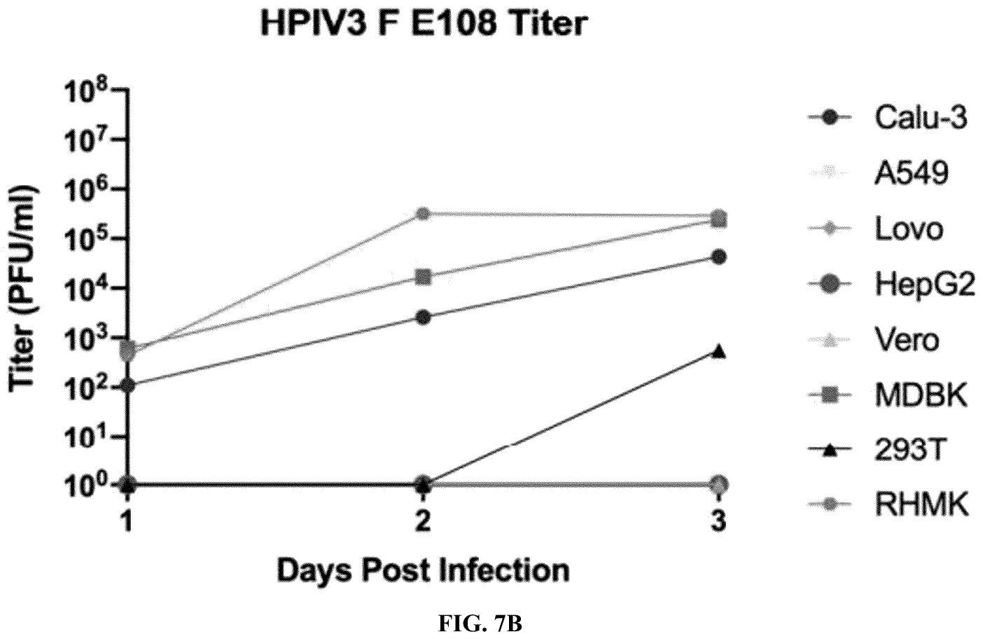

In the case of HPIV3, our laboratory demonstrated that the F protein of circulating strains consistently contains a monobasic cleavage motif (R-X-E-R) with glutamate at site 108, and that the dibasic cleavage motif is an artifact of viral propagation in cell culture (25). Cell lines classically used for viral isolation, such as Rhesus Monkey Kidney (RHMK) and Madin-Darby Bovine Kidney (MDBK) cells, are able to cleave F0 and propagate circulating HPIV3; however, most cultured human cell lines lack the requisite host protease to release infectious virions (FIG. 7) (25). Although cells lacking the protease for F0 cleavage are susceptible to primary infection by virions activated by permissive cells or exogenous trypsin, HPIV3 F with E108 rapidly mutates to K108 when cultured in these models, suggesting an absence of the selective pressures that limit this feature of lab adaptation in the circulating strains.

Rapid evolution of the HPIV3 fusion complex: HPIV3 isolated directly from human subjects bear HN-F fusion complexes that are highly specialized for propagation in their natural human host environment. Mutations rapidly emerge that confer fitness specific to the cell line and culture conditions applied. The mutations in HN and F result in distinct characteristics between the fusion machinery of clinical isolates (CI) and lab-adapted (LA) strains (25, 26). Features that confer greater fusogenicity and success in monolayer cell culture, including high avidity, low neuraminidase activity, and highly triggerable F, are maladaptive; and LA strains are frequently not viable in vivo (25-27). Our laboratory has identified authentic lung models, including lung organoids and human airway epithelia (HAE) grown at an air-liquid interface, in which CI HPIV3 strains can grow efficiently and without selection for mutations favored in cell culture (25, 26, 28, 29). Authentic lung models are critical for investigating the host factors responsible for cleaving HPIV3 F in the human lung and exploring the impact of their expression on viral tropism.
A.3. What are the consequences of furin-independent HPIV3 F cleavage in the human lung? Dependence on cell type-specific proteases further regulates infection. As an example, the fusion or spike proteins of furin-independent viruses (including Sendai virus, human metapneumovirus, SARS-COV, and SARS-COV-2) can be cleaved by transmembrane proteases expressed on their host or target cells (30-33). Cleavage by host cells can facilitate the release of infectious virions with active fusion proteins, and target cells bearing the necessary extracellular proteases can activate virions with uncleaved fusion proteins. The latter case may be particularly important for the spread of HPIV3.

Viruses, including HPIV3 and influenza, have surface glycoproteins that bind sialic acid and tether the virions to target cells (34, 35). Human lung cells release sialic acid-bearing mucins that can mimic cell surface receptors and bind virion glycoproteins (36). Both HPIV3 and influenza circumvent this sequestration by cleaving sialic acids with their HN or neuraminidase (NA) surface glycoproteins, respectively (37). Unlike influenza, HPIV3 HN binding to sialic acid is sufficient to cause HN to trigger F to extend and irreversibly fold into its post fusion state.

When HN is bound to sialic acid on host cell mimic, the resultant F triggering constitutes an irreversible loss of finite fusion protein on the surface of the virion (38-40). Circulating strains of HPIV3 bearing F0 on their surface may be more resistant to inactivation by untimely F triggering. As virions traverse mucus in the lung, inactive F0 may remain dormant until HN binds a target cell bearing a surface protease that can cleave F0, at which point the activated F protein may be able to insert into the target cell and facilitate fusion. This theorized role of F0 represents a novel mechanism for evading premature fusion protein triggering induced by mucins released in authentic models and human lung.
A.4. Scientific premise: F processing as a critical regulator of viral tropism in the human lung. The objective of this application is to characterize HPIV3 F processing in the human lung and elucidate the functional role of F cleavage as an unexplored regulator of viral fusion and a determinant of viral tropism. F0 cleavage was thought to be executed by the ubiquitously expressed furin because F0 contains a dibasic cleavage motif (22). However, we found that the dibasic cleavage motif R-X-K-R, which is the foundation of this assumption, is actually an artifact of laboratory adaptation. In contrast, circulating strains of HPIV3 have a monobasic cleavage motif R-X-E-R, which is cleaved by a much narrower population of cells expressing a yet unidentified protease (25). Once the protease is identified, the locations and mechanisms of F0 cleavage may be examined in authentic lung models.

Disentanglement of F0 activation and furin radically alters the role of F from a protein that is activated by nearly all cells, to one that only facilitates infection when: (1) released by cells with the necessary proteases for cleavage, or (2) the virion comes into contact with proteases that can facilitate cleavage in the environment or on target cells. By studying circulating strains of HPIV3 in authentic lung models, we maintain selective pressures favoring clinically relevant features of HPIV3 fusion machinery and are able to hone in on host factors like the proteases responsible for F0 cleavage in the human lung. The candidate proteases that we propose are responsible for F0 cleavage were selected for their expression in authentic lung models and cells capable of releasing infectious virions. The majority of these proteases are either membrane-bound or secreted and would therefore support F0 cleavage on the cell surface or extracellularly. During the execution of this grant, we will test our hypothesis that F0 is cleaved by transmembrane proteases and examine how this activation requirement may affect viral tropism in the human lung.

### B. Approach

B.0.1 Rigor and transparency. All experiments will be performed with appropriate biological and technical replicates, and repeated on separate days. Orthogonal assays will be performed to confirm results. Positive and negative controls (e.g., infected vs. non-infected, treated vs. untreated) are included in all experiments. Biological and chemical resources will be validated as outlined in the “Authentication” document. Detailed experimental protocols and primary data will be made available to interested parties. Members of our laboratory have expertise in the virology, cell biology, and molecular biology techniques outlined, and are readily available to provide guidance. We review all data prior to analysis, and data will not be excluded.
B.0.2 Panel of biochemical, functional assays, and models.

Cell-to-cell fusion assays: The impact of host factors on cell-to-cell fusion mediated by HPIV3 HN and F is assessed by co-transfecting viral and host proteins with β-galactosidase alpha or omega peptides. Complementation of the β-galactosidase peptides is quantified by measuring luminescence from a chemiluminescent substrate cleaved by the complete enzyme (26, 29).

Dissecting stages of infection: Stages of HPIV3 infection can be regulated with a combination of altered temperatures and treatments. At 4oC, HN are able to bind to target cells, but F triggering and fusion require temperatures near 37oC. Zanamivir, a receptor analog, can be added to prevent HN binding to target cells by blocking HN's sialic acid binding to site I (41).

Human Airway Epithelial (HAE) cultures: MatTek EpiAirway AIR-100 system—normal human-derived tracheal/bronchial epithelial cells cultured at an air-liquid interface with polarized 3D structures consisting of basal cells, mucus producing goblet cells, functional tight junctions, and beating cilia (27).

Lung organoids (LO): human pluripotent stem cells differentiated into branching airways that terminate in alveoli. The proposed lung organoids contain cells with markers for airway basal, ciliated, secretory, goblet, and neuroendocrine cells, as well as alveolar type I and type II cells (28, 42).
B.1. Aim 1: What host factors are required for circulating human parainfluenza virus 3 fusion protein cleavage and successful viral entry?
B1.1. Hypothesis and Rationale: The Moscona-Porotto laboratory's previous work genotyping circulating strains of HPIV3 demonstrated that the virus sequenced directly from patients, without passaging in cell culture, consistently contained fusion proteins bearing glutamic acid at site 108 (E108). Further, this feature was maintained during passaging in authentic lung models including primary human airway epithelial cells and lung organoids (28).

However, HPIV3 with F bearing lysine at site 108 (K108) rapidly emerges in monolayer cell culture models, thereby creating a dibasic cleavage site (R-X-K-R) and enabling F0 to be cleaved by ubiquitously expressed furin. Although F0 contains the theoretical minimal furin cleavage site R-X-X-R, the proprotein convertase is not sufficient to cleave HPIV3 F bearing E108 in most immortalized cell lines (25, 43).

We hypothesized that HPIV3 fusion protein cleavage in the human lung is not furin dependent and is instead facilitated by transmembrane proteases. The observation that furin is neither necessary nor sufficient for F activation of circulating HPIV3 highlights an additional regulatory constraint for the viral fusion machinery.

Elucidating the critical protease or proteases enables identification of specific cell types with the capacity to activate HPIV3 F and support mechanistic studies describing where this critical step for viral fusion is achieved. We screen candidate proteases expressed in the permissive cells in order to determine which may allow non-permissive cells to produce functional virions. Further, we assess if F0 cleaving proteases cleave F0 during viral entry or primarily during viral egress. Lastly, we evaluate if human lung cell susceptibility to HPIV3 infection is predicted by cell-specific protease expression. Taken together, the results identify the necessary proteases for HPIV3 spread and their contribution for viral tropism in the human lung.
B.1.2 Relevant Preliminary Data

We established that CI HPIV3 propagated in authentic lung models (including HAE or lung organoids) are under minimal selective pressure and maintain the R-X-E-R cleavage motif (25, 28). Key host factors for growing these clinical isolates in the authentic models are host proteases that cleave HPIV3 F bearing E108, thereby enabling multicycle replication. Based on limited viral spread and the absence of cleaved F from virions released by HepG2,HEK 293T, and A549 cells, we hypothesized that the F E108-cleaving protease is expressed in the authentic lung models, but not in these immortalized human cell lines.

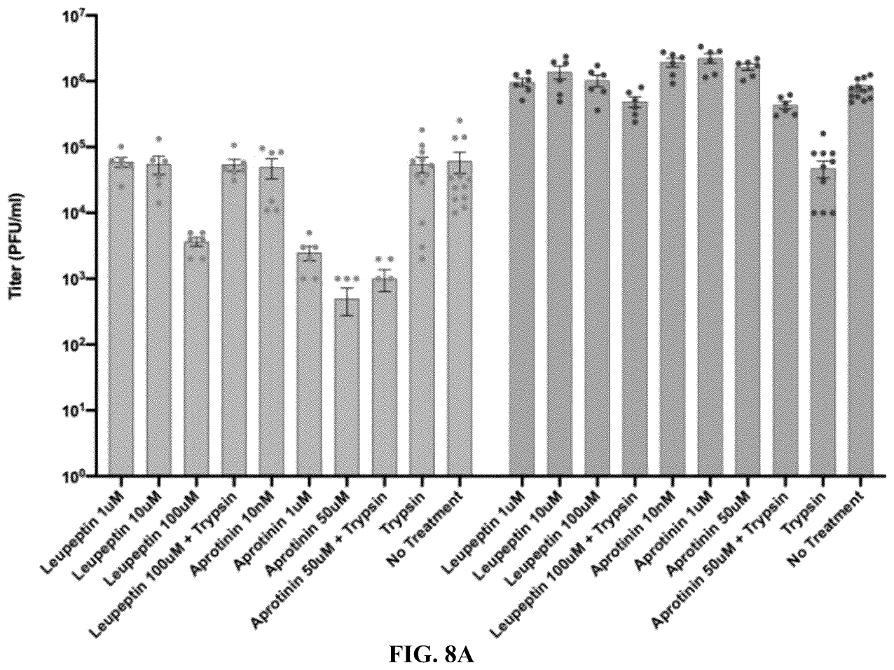

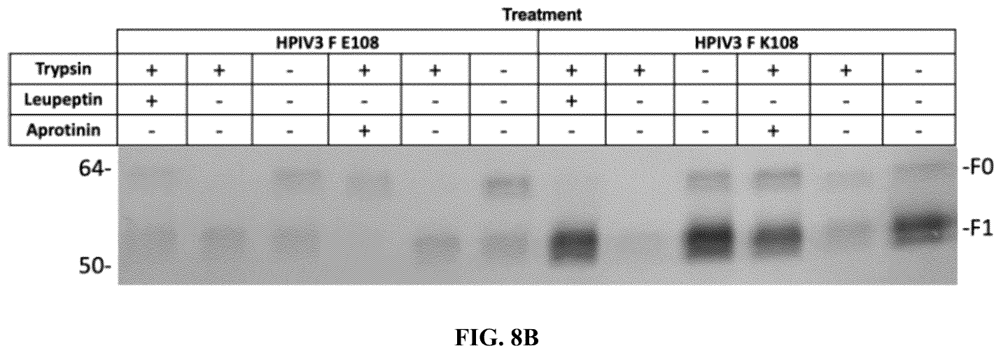

We therefore compared RNA sequencing data from permissive (HAE, Calu-3) and non-permissive (HepG2, HEK 293T, A549) cultures to identify candidate proteases that confer the ability to cleave F E108 and propagate infectious CI HPIV3 (Table 1) (1). Based on the observation that the majority of the candidate proteases are membrane-bound serine proteases, we aimed to test whether the cell impermeable serine protease inhibitors (leupeptin and aprotinin) could inhibit F0 cleavage and the release of infectious virions from Calu-3 cells. We observed a dose-dependent reduction in viral titer and significant reduction in F0 cleavage with aprotinin treatment, suggesting that the F0-cleaving protease is a membrane-bound or secreted serine protease (FIG. 8).

TABLE 1
RNA-seq candidate protease transcript expression levels in CI HPIV3
permissive and non-permissive cells. Depicted values denote the
transcripts per million (TPM) of detected genes with TPM >5 in
permissive cells and <5 in non-permissive cells (1).

| Gene Symbol | HAE | Calu-3 | A549 | HEK293T | HEP G2 |
|---|---|---|---|---|---|
| TMPRSS4 | 246 | 29 | 1 | 0 | 1 |
| ST14 | 87 | 70 | 1 | 0 | 2 |
| PRSS22 | 143 | 50 | 1 | 0 | 1 |
| KLK10 | 63 | 10 | 1 | 1 | 1 |
| PRSS8 | 63 | 57 | 1 | 0 | 4 |
| KLK11 | 103 | 6 | 1 | 0 | 1 |
| ADAM28 | 36 | 20 | 1 | 0 | 1 |
| KLK6 | 2 | 108 | 1 | 0 | 1 |
| ADAM8 | 5 | 13 | 2 | 4 | 1 |
| TMPRSS2 | 32 | 3 | 1 | 0 | 4 |
| Furin | 17 | 15 | 23 | 24 | 22 |

B.1.3. Proposed Experiments
B.1.3.a. Assess HPIV3 F cleavage and viral propagation in the presence of candidate proteases: We will perform a gain-of-function screen to identify human proteases that are sufficient to cleave HPIV3 with F bearing E108. HEK 293T cells will be transfected with open reading frame (ORF) or CRISPR Activation (CRISPRa) plasmids for each candidate protease. The transfected cells will be infected in parallel with either HPIV3 F E108 or HPIV3 F K108 (Both viruses are engineered with an mCherry marker). Following transfection, candidate protease expression is confirmed with RT-PCR and western blotting. HEK 293T cells that have been transfected with empty vector or non-specific 20 nt guide 15 RNA (gRNA) are utilized as the HPIV3 F E108 non-permissive negative controls. As a positive control, HPIV3 F K108 infections are performed in parallel with all experiments to confirm that effects on F protein cleavage and viral propagation are F E108-specific. Transfected cells are assessed for their ability to promote multicycle replication of HPIV3 F E108, release of trypsin-independent infectious virions, and F0 cleavage. Proteins that confer these functions are sufficient to enable HEK 293T cells to propagate HPIV3 F E108.

The necessity for identified proteases for HPIV3 F cleavage in authentic lung models and permissive cells will be assessed in HAE (MatTek), LO, and Calu-3 cell (ATCC) culture models. Lung models are transduced with lentiviruses carrying Cas9 and sgRNA designed to knock out each candidate protease. If multiple proteases are identified to be sufficient, combinations of proteases will be knocked out via co-transduction with multiple lentiviruses using sgRNA specific to each protease. Following transduction and confirmation of protein-level knockout, the cells' abilities to cleave F E108 and produce infectious virions will be assessed. Identification of protease knockouts that lead to critical loss of HPIV3 F E108 cleavage functionality in each of these models provides insights about the spectrum of host proteases that contribute to HPIV3 F processing in the human lung.
B.1.3.b. Characterizing the cleavage of HPIV3 F E108 at the surface of permissive cells: Inhibition of F E108 cleavage by cell impermeable protease inhibitors suggests that the proteases responsible for F cleavage are acting extracellularly, independent of the furin-dependent cleavage in the trans-Golgi network identified for F K108 (44). We characterize the F cleavage conditions that favor successful infection by HPIV3 F E108. Cell-cell fusion assays are employed to evaluate the interaction of proteases with HPIV3 F. First, cis-activation of F will be assessed by co-transfecting HEK 293T cells bearing HPIV3 HN and F E108 with a candidate protease, and then assessing if the protease expressed on the same membrane as the HN-F complex is able to cleave F0 and enable the complex to facilitate cell-cell fusion. Trans-activation of F will be assessed by transfecting target cells with the candidate proteases, followed by quantitation of cell-cell fusion between protease-expressing cells and HN-F (E108) expressing cells. If trans-activation of F is observed, then the experiment is repeated in the presence of Zanamivir to assess if HN tethering of viral fusion machinery to the protease bearing target cell membrane is necessary for F cleavage, or increases the likelihood that cleavage will occur. Investigating cis- and trans-protease activity will clarify which proteases are able to cleave F0 during viral egress and which may cleave F0 on virions at the surface of target cells (30).
B.1.3.c. Evaluate the contributions of F cleaving proteases to viral tropism: HAE and LO consist of heterogeneous cell populations with distinct expression profiles and resultant variable susceptibility to primary infection (28). We ascertain if the expression levels of sufficient proteases correlate with increased viral entry. HPIV3 F E108 bearing only F0 will be incubated with HAE and LO. Associations between protease expression and susceptibility to primary infection will be quantitated by dissociating the tissues, staining with cell specific markers, and measuring the proportion of infected cells within each population using flow cytometry. The results of these experiments will provide insights about cell types most susceptible to HPIV3 infection and the contributions of host proteases to their susceptibility.
B.1.4. Potential pitfalls and alternative approaches
a. There are hundreds of proteases expressed in the human lung and multiple may contribute to HPIV3 F E108 cleavage. The expression-based approach utilized for identifying candidate proteases may not identify acutely expressed proteases (45). A CRISPRa protease library screen could be performed to evaluate the ability of every protease in human genome to cleave clinical isolate HPIV3 F and facilitate propagation of the virus.
b. HEK 293T cells may not properly express, modify, or transport proteases introduced with exogenous genes. Protease mRNA expression levels will be compared to HAE and LO expression levels. Western blotting and surface biotinylation will be employed to confirm the size and localization of the transfected proteases.
c. Primary infection by HPIV3 bearing only F0 will be dependent on secreted or trans-acting proteases to cleave F and facilitate virion-cell fusion. If the protease is secreted or the virus is released after F0 cleavage by a trans-acting protease, then cells that do not express the F0 cleaving protease may be infected by the virions bearing cleaved F. Infection of these cells may confound efforts to correlate protease expression with susceptibility to primary infection. Following inoculation, cells are incubated in the presence of Zanamivir, an HPIV3 HN receptor analog that inhibits viral spread, to prevent subsequent cycles of infection.
B.1.5. Anticipated outcome: Aim 1 will identify which proteases are able to facilitate F E108 cleavage and the production of infectious virions. The outlined experiments will identify host factors essential for successful propagation of HPIV3 F bearing E108. Identifying the cell types expressing these key proteases will inform which cells in vivo are most receptive to primary infection by F0 bearing virions and responsible for propagation of infectious viruses. The complementary mechanistic studies will demonstrate if HPIV3 F is only cleaved upon egress or if viruses bearing F0 may infect surface protease bearing cells. Identifying proteases responsible for cleaving HPIV3 F will shed light on where and at what stage of the viral life cycle this essential cleavage event occurs in human hosts.
B.2. Aim 2: Through what mechanisms does F cleavage by host proteases confer increased fitness in the human lung?
B.2.1. Hypothesis and Rationale: Although viruses bearing the CI or LA Fare viable in authentic lung models, the absence of the LA F cleavage motif in circulating strains of HPIV3 suggest there are evolutionary forces in the human lung favoring the conservation of the CI cleavage motif (24, 25). It is not yet known if HPIV3 bearing F with the CI cleavage motif is able to outcompete virions bearing the LA cleavage motif in authentic lung models. The persistence of the CI F cleavage motif may be explained by improved fusion efficiency with F0 cleaved during fusion or F0 resistance to irreversible inactivation as virions traverse the host environment.

We hypothesized that F0 on the surface of virions is evolutionarily advantageous due to more efficient viral fusion when F is cleaved at the target cell surface, and increased resistance of F0 to inactivation by host receptor mimics. First, we perform co-infections with circulating and furin-dependent strains of HPIV3 to assess if circulating strains are dominant in authentic lung models. Next, we evaluate the efficiency of infections facilitated by F0 cleavage by host cell proteases at the cell surface at the time of infection. Lastly, we determine if the inactivity of F0 enables virions to evade virucidal pre-triggering of F and preserve viable fusion peptides until the F protein can be cleaved by proteases at the surfaces of target cells.

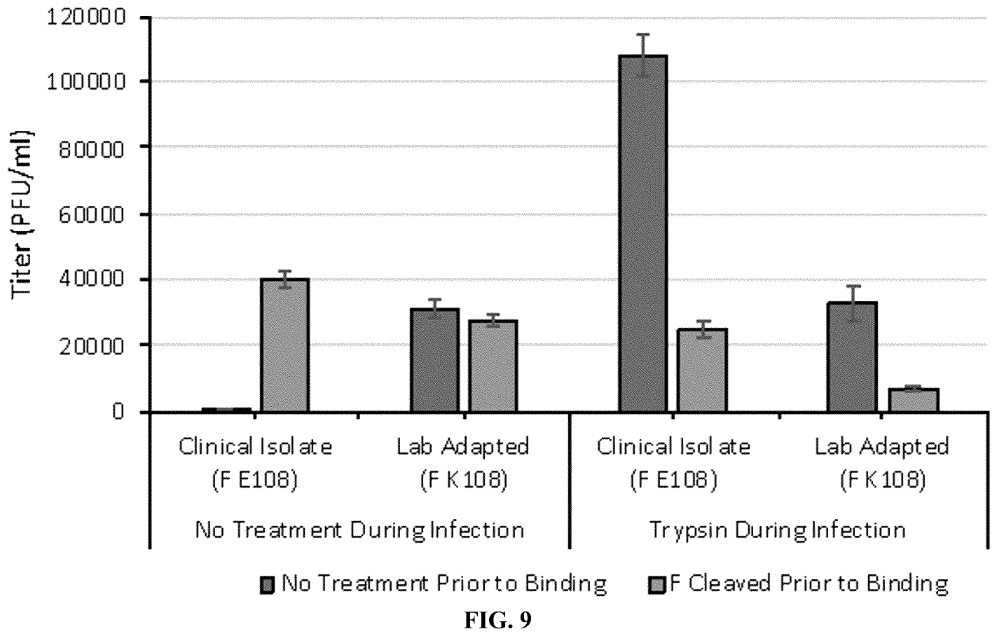

In this aim, we determine if F bearing the clinical isolate cleavage motif on the surface of virions confers fitness benefits in the human lung, and evaluate if this advantage is attributable to F0 facilitating more efficient fusion or its capacity to delay activation until reaching the surface of its target cell.
B.2.2. Relevant preliminary data: In the experiment reported in FIG. 9, HPIV3 bearing F0 or cleaved F was bound to cells and then allowed to infect in the presence or absence of the F-cleaving exogenous protease TPCK-treated trypsin. Relative to virions with F cleaved prior to binding, HPIV3 bearing F0 infected significantly more cells when F0 was cleaved while the virion was bound to its target cell. These results suggest that F0 on the surface of virions are not functionless products of inefficient viral protein processing, but rather may facilitate efficient infection of target cells expressing F-cleaving proteases (30).
B.2.3. Experiments
B.2.3.a. Does the clinical isolate cleavage motif confer an evolutionary advantage over the lab adapted motif in authentic lung models?: We assess the competitive fitness of HPIV3 F bearing E108 (CI) with HPIV3 F bearing K108 (LA) to determine if either virus has a competitive advantage in authentic lung models. (i) HAE and LO will be infected with equal titers of HPIV3 F E108 or HPIV F K108 with otherwise identical viral genomes to assess the rate of viral spread and clearance in each authentic lung model. Cells from each infection condition will be collected for RNA sequencing prior to infection, three days post infection, and following viral clearance to assess differentially expressed genes. (ii) HAE and LO will be co-infected with varying proportions (1:100, 1:10, 1:1, 10:1, and 100:1) of HPIV3-mCherry F E108 and HPIV3-eGFP F K108. Each day post-infection, the proportion of mCherry and eGFP positive cells will be compared in order to monitor the spread of each virus. Viral supernatants will also be collected and titered daily from 1-7 days post-infection to monitor shifts in the viral populations. In order to ensure that any observed dominance is not a result of the fluorescent markers, this experiment will also be performed with HPIV3-eGFP F E108 and HPIV3-mCherry F K108. The results of this sub aim will demonstrate if HPIV3 bearing the CI F cleavage motif is more fit for growth in authentic lung models, and thus able to maintain or gain dominance during coinfection with lab adapted virus.
B.2.3.b. Does F0 facilitate more efficient fusion when cleaved on a target cell-bound virion?: Based on the results in FIG. 9, virions may achieve more efficient infection when F0 is cleaved while the virion is receptor-bound to the target cell. Clinical isolate HPIV3 bearing F0 or cleaved F will be incubated with the LO, HAE, and gain-of-function protease-expressing cells at 4oC to allow virion-target cell binding. Following binding, unbound viruses will be removed and viral fusion will be allowed to progress by incubating the cells at 37oC. In parallel with these conditions, TPCK-treated trypsin positive controls will have the exogenous protease added during the 37oC viral fusion incubation step. Performing this assay in the gain-of-function cultures will provide insight as to whether more efficient infection is facilitated by all identified proteases, or if the effect is protease-specific. Further, more efficient infection of LO and HAE by pre-bound viruses bearing F0 would suggest that infection in developmental and/or adult human lungs is promoted by a fusion cascade in which cleavage occurs at the surface of the target cells.
B.2.3.c. Assess F0 resistance to host cell mimicry induced premature activation: To assess premature F activation by secreted mucins, we first apply cell-based assays to correlate conformational changes in HPIV3 F with fusogenicity. HEK 293T cells will be transfected with HPIV3 HN, and either F E108 (CI, uncleaved F) or HPIV3 F K108 (LA, cleaved F). The cells will then be treated with neuraminidase (to deplete surface cell receptors) and cycloheximide (to halt protein expression). Neuraminidase is removed and the cells exposed to lung cell secretome solutions (HAE or Calu-3 ALI apical washes) (46). The proportions of F in the pre-fusion and post-fusion (post-triggering) states will be quantified with immunohistochemistry using conformation-specific HPIV3 F antibodies (40).

Next, the functional implications of mucin-induced F triggering will be assessed by quantifying HN-F and HN-F0 mediated cell-to-cell fusion. For both conditions, fusion with target cells will be assessed in the presence of TPCK-treated trypsin or with target cells expressing trans-acting proteases identified in aim 1, so viable F0 may be cleaved, and fusion can be quantified. The F-triggering components within the secretomes will be selectively isolated by HN binding affinity and identified by proteomic mass spectrometry.

Finally, F-triggering host factors are systematically overexpressed and knocked out in Calu-3 cells to assess their capacity to inhibit HPIV3 bearing F0 or cleaved F. The results of this study elucidate host factors that hinder HPIV3 infection by binding HN and triggering F, and demonstrate if F0 is resistant to the potentially virucidal binding.
B.2.4. Potential pitfalls and alternative approaches
a. It was possible that no difference between infection with the two viruses would be observed. No significant differences in viral spread, viral clearance, or dominance of either virus in authentic lung models would suggest selection for HPIV3 F bearing E108 (CI) does not occur at the cellular level and instead may relate to other pressures only present at the organismal level. Comparing the spread of each mutant in cotton rats or ferret models is used to assess the fitness of each mutant in vivo (47, 48).
b. The data in FIG. 9 may also be interpreted to indicate that F0 on the surface of virions may be more stable than cleaved F. Virions bearing only F0 or cleaved F can be incubated at physiological temperatures (37oC) and collected at various intervals to assess loss of titer over time as an indicator of virion degradation. TPCK-treated trypsin would be added during the infection step of titering both viruses so the F0 bearing virions will be able to infect the Vero cells. If F degradation is the rate determining step for loss of virion viability, this experiment would quantify differences between virions bearing F0 and cleaved F.
c. It is currently unknown if HAE or Calu-3 cells secrete sialic acid bearing proteins that can trigger HPIV3 F. If F triggering activity is not observed, then the results of the HN affinity protein isolation and proteomic mass spectrometry could still identify host factors that hinder the spread of HPIV3 by competing with target cell receptors. It is also possible that F is triggered by HN binding to membrane-bound sialic acid bearing proteins like MUC1. Such an event could be observed by incubating HPIV3 virions in the presence of HAE or Calu-3 cells and staining for post-fusion F. If pre-triggering occurs near the surface of the cell but fusion is not achieved, then the post-fusion F may be detectable on virions bound to surface-anchored mucins.
B.2.5. Anticipated outcome: Aim 2 will determine if HPIV3 with F bearing the clinical isolate cleavage motif has an evolutionary advantage in the human lung as a result of more efficient and selective activation of F at the surface of target cells. B.2.3.a will compare the spread and clearance of HPIV3 bearing F with the CI or LA cleavage motif to determine if selective pressures favoring the pervasiveness of CI F can be observed in authentic lung models. B.2.3.c will determine if F0 on the surface of virions is able to exploit surface proteases and facilitate more efficient infection of target cells. Lastly, B.2.3.c will determine if F0 enables viruses to circumvent virucidal interactions with target receptor mimics. Taken together, the results of this aim will illustrate if the circulating HPIV3 cleavage motif is conserved in the human lung because F0 confers the unique benefits of resisting inactivation in transit to target cells and efficiently fusing upon activation at their surface.

It is to be understood that the compositions and methods for treating or preventing HPIV3 are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

### CITATIONS

1. DepMap CCLE_expression [Internet]. 2022.
2. Wang X, Li Y, Deloria-Knoll M, Madhi SA, Cohen C, Ali A, et al. Global burden of acute lower respiratory infection associated with human metapneumovirus in children under 5 years in 2018: a systematic review and modelling study. Lancet Glob Health. 2021; 9(1):e33-c43. Epub 2020/11/30. doi: 10.1016/S2214-109X(20)30393-4. PubMed PMID: 33248481; PubMed Central PMCID: PMCPMC7783516.
3. Park GYS, Tishkowski K. Paramyxovirus. StatPearls. Treasure Island (FL)2022.
4. Loughlin GM, Moscona A. The cell biology of acute childhood respiratory disease: therapeutic implications. Pediatr Clin North Am. 2006;53 (5):929-59. PubMed PMID: 17027618.
5. Moscona A. Entry of parainfluenza virus into cells as a target for interrupting childhood respiratory disease. J Clin Invest. 2005;115 (7):1688-98. PubMed PMID: 16007245.
6. Bjornson CL, Johnson DW. Croup. Lancet. 2008; 371(9609):329-39. Epub 2008/02/26. doi: 10.1016/S0140-6736(08)60170-1. PubMed PMID: 18295000.
7. DeLaMora P, Moscona A. A daring treatment and a successful outcome: the need for targeted therapies for pediatric respiratory viruses. Pediatric transplantation. 2007; 11(2):121-3. PubMed PMID: 17300487.
8. Scheid A, Choppin PW. Isolation and purification of the envelope proteins of Newcastle disease virus. J Virol. 1973; 11(2):263-71. Epub 1973/02/01. doi: 10.1128/JVI.11.2.263-271.1973. PubMed PMID: 4734650; PubMed Central PMCID: PMCPMC355091.
9. Scheid A, Choppin PW. Identification of biological activities of paramyxovirus glycoproteins. Activation of cell fusion, hemolysis, and infectivity of proteolytic cleavage of an inactive precursor protein of Sendai virus. Virology. 1974; 57(2):475-90. Epub 1974/02/01. doi: 10.1016/0042-6822(74)90187-1. PubMed PMID: 4361457.
10. Porotto M, Palmer SG, Palermo LM, Moscona A. Mechanism of fusion triggering by human parainfluenza virus type III: communication between viral glycoproteins during entry. J Biol Chem. 2012; 287(1):778-93. doi: 10.1074/jbc.M111.298059. PubMed PMID: 22110138; PubMed Central PMCID: PMC3249132.
11. Yin HS, Paterson RG, Wen X, Lamb RA, Jardetzky TS. Structure of the uncleaved ectodomain of the paramyxovirus (hPIV3) fusion protein. Proc Natl Acad Sci U S A. 2005; 102 (26): 9288-93. PubMed PMID: 15964978.
12. Yin HS, Wen X, Paterson RG, Lamb RA, Jardetzky TS. Structure of the parainfluenza virus 5 F protein in its metastable, prefusion conformation. Nature. 2006; 439(7072):38-44. PubMed PMID: 16397490.
13. Palgen JL, Jurgens EM, Moscona A, Porotto M, Palermo LM. Unity in diversity: shared mechanism of entry among paramyxoviruses. Prog Mol Biol Transl Sci. 2015; 129:1-32. doi: 10.1016/bs.pmbts.2014.10.001. PubMed PMID: 25595799; PubMed Central PMCID: PMC4369139.
14. Harrison SC. Viral membrane fusion. Nature structural & molecular biology. 2008; 15(7):690-8. PubMed PMID: 18596815.
15. Chang A, Dutch RE. Paramyxovirus fusion and entry: multiple paths to a common end. Viruses. 2012; 4(4):613-36. Epub 2012/05/17. doi: 10.3390/v4040613. PubMed PMID: 22590688; PubMed Central PMCID: PMC3347325.
16. Lamb RA, Paterson RG, Jardetzky TS. Paramyxovirus membrane fusion: lessons from the F and HN atomic structures. Virology. 2006; 344(1):30-7. PubMed PMID: 16364733.
17. Plemper RK, Brindley MA, Iorio RM. Structural and mechanistic studies of measles virus illuminate paramyxovirus entry. PLOS Pathog. 2011; 7(6):e1002058. Epub 2011/06/10. doi: 10.1371/journal.ppat.1002058. PubMed PMID: 21655106; PubMed Central PMCID: PMC3107210.
18. White JM, Delos SE, Brecher M, Schornberg K. Structures and mechanisms of viral membrane fusion proteins: multiple variations on a common theme. Critical reviews in biochemistry and molecular biology. 2008; 43(3):189-219. PubMed PMID: 18568847.
19. Sapir A, Avinoam O, Podbilewicz B, Chernomordik LV. Viral and developmental cell fusion mechanisms: conservation and divergence. Dev Cell. 2008; 14(1):11-21. Epub 2008/01/16. doi: 10.1016/j.devcel.2007.12.008. PubMed PMID: 18194649.
20. Smith EC, Popa A, Chang A, Masante C, Dutch RE. Viral entry mechanisms: the increasing diversity of paramyxovirus entry. Febs J. 2009; 276(24):7217-27. Epub 2009/11/03. doi: 10.1111/j.1742-4658.2009.07401.x. PubMed PMID: 19878307; PubMed Central PMCID: PMC2795005.
21. Lee B, Ataman ZA. Modes of paramyxovirus fusion: a Henipavirus perspective. Trends Microbiol. 2011; 19(8):389-99. Epub 2011/04/23. doi: 10.1016/j.tim.2011.03.005. PubMed PMID: 21511478.
22. Ortmann D, Ohuchi M, Angliker H, Shaw E, Garten W, Klenk HD. Proteolytic cleavage of wild type and mutants of the F protein of human parainfluenza virus type 3 by two subtilisin-like endoproteases, furin and Kex2. J Virol. 1994; 68(4):2772-6. Epub 1994/04/01. doi: 10.1128/JVI.68.4.2772-2776.1994. PubMed PMID: 8139055; PubMed Central PMCID: PMCPMC236759.
23. Klenk HD, Garten W. Host cell proteases controlling virus pathogenicity. Trends Microbiol. 1994; 2(2):39-43. Epub 1994/02/01. doi: 10.1016/0966-842x(94)90123-6. PubMed PMID: 8162439.
24. Coelingh KV, Winter CC. Naturally occurring human parainfluenza type 3 viruses exhibit divergence in amino acid sequence of their fusion protein neutralization epitopes and cleavage sites. J Virol. 1990; 64(3):1329-34. Epub 1990/03/01. doi: 10.1128/JVI.64.3.1329-1334.1990. PubMed PMID: 1689394; PubMed Central PMCID: PMCPMC249251.
25. Palermo LM, Uppal M, Skrabanek L, Zumbo P, Germer S, Toussaint NC, et al. Features of Circulating Parainfluenza Virus Required for Growth in Human Airway. mBio. 2016; 7(2):e00235. Epub 2016/03/17. doi: 10.1128/mBio.00235-16. PubMed PMID: 26980833; PubMed Central PMCID: PMC4807361.
26. Iketani S, Shean RC, Ferren M, Makhsous N, Aquino DB, des Georges A, et al. Viral Entry Properties Required for Fitness in Humans Are Lost through Rapid Genomic Change during Viral Isolation. MBio. 2018; 9(4). doi: 10.1128/mBio.00898-18. PubMed PMID: 29970463; PubMed Central PMCID: PMC6030562.
27. Palermo L, Porotto M, Yokoyama C, Palmer S, Mungall B, Greengard O, et al. Human parainfluenza virus infection of the airway epithelium: the viral hemagglutinin-neuraminidase regulates fusion protein activation and modulates infectivity. J Virol. 2009; 83(13):6900-8. doi: 10.1128/JVI.00475-09. PubMed Central PMCID: PMC2698534.
28. Porotto M, Ferren M, Chen YW, Siu Y, Makhsous N, Rima B, et al. Authentic Modeling of Human Respiratory Virus Infection in Human Pluripotent Stem Cell-Derived Lung Organoids. MBio. 2019; 10(3). doi: 10.1128/mBio.00723-19. PubMed PMID: 31064833; PubMed Central PMCID: PMC6509192.
29. Palmer SG, DeVito I, Jenkins SG, Niewiesk S, Porotto M, Moscona A. Circulating clinical strains of human parainfluenza virus reveal viral entry requirements for in vivo infection. J Virol. 2014; 88(22):13495-502. doi: 10.1128/JVI.01965-14. PubMed PMID: 25210187; PubMed Central PMCID: PMC4249073.
30. Glowacka I, Bertram S, Muller MA, Allen P, Soilleux E, Pfefferle S, et al. Evidence that TMPRSS2 activates the severe acute respiratory syndrome coronavirus spike protein for membrane fusion and reduces viral control by the humoral immune response. J Virol. 2011; 85(9):4122-34. Epub 2011/02/18. doi: 10.1128/JVI.02232-10. PubMed PMID: 21325420; PubMed Central PMCID: PMCPMC3126222.
31. Zang R, Gomez Castro MF, McCune BT, Zeng Q, Rothlauf PW, Sonnek NM, et al. TMPRSS2 and TMPRSS4 promote SARS-COV-2 infection of human small intestinal enterocytes. Sci Immunol. 2020; 5(47). Epub 2020/05/15. doi: 10.1126/sciimmunol.abc3582. PubMed PMID: 32404436; PubMed Central PMCID: PMCPMC7285829.
32. Shirogane Y, Takeda M, Iwasaki M, Ishiguro N, Takeuchi H, Nakatsu Y, et al. Efficient multiplication of human metapneumovirus in Vero cells expressing the transmembrane serine protease TMPRSS2. J Virol. 2008; 82(17):8942-6. Epub 2008/06/20. doi: 10.1128/JVI.00676-08. PubMed PMID: 18562527; PubMed Central PMCID: PMCPMC2519639.
33. Abe M, Tahara M, Sakai K, Yamaguchi H, Kanou K, Shirato K, et al. TMPRSS2 is an activating protease for respiratory parainfluenza viruses. J Virol. 2013; 87(21):11930-5. Epub 2013/08/24. doi: 10.1128/JVI.01490-13. PubMed PMID: 23966399; PubMed Central PMCID: PMCPMC3807344.
34. Suzuki T, Portner A, Scroggs RA, Uchikawa M, Koyama N, Matsuo K, et al. Receptor specificities of human respiroviruses. J Virol. 2001; 75(10):4604-13. Epub 2001/04/20. doi: 10.1128/JVI.75.10.4604-4613.2001. PubMed PMID: 11312330; PubMed Central PMCID: PMCPMC114213.
35. Connor RJ, Kawaoka Y, Webster RG, Paulson JC. Receptor specificity in human, avian, and equine H2 and H3 influenza virus isolates. Virology. 1994; 205(1):17-23. Epub 1994/11/15. doi: 10.1006/viro.1994.1615. PubMed PMID: 7975212.
36. Barnard KN, Alford-Lawrence BK, Buchholz DW, Wasik BR, LaClair JR, Yu H, et al. Modified Sialic Acids on Mucus and Erythrocytes Inhibit Influenza A Virus Hemagglutinin and Neuraminidase Functions. J Virol. 2020; 94(9). Epub 2020/02/14. doi: 10.1128/JVI.01567-19. PubMed PMID: 32051275; PubMed Central PMCID: PMCPMC7163148.
37. Cohen M, Zhang XQ, Senaati HP, Chen HW, Varki NM, Schooley RT, et al. Influenza A penetrates host mucus by cleaving sialic acids with neuraminidase. Virol J. 2013; 10:321. Epub 2013/11/23. doi: 10.1186/1743-422X-10-321. PubMed PMID: 24261589; PubMed Central PMCID: PMCPMC3842836.
38. Farzan SF, Palermo LM, Yokoyama CC, Orefice G, Fornabaio M, Sarkar A, et al. Premature Activation of the Paramyxovirus Fusion Protein before Target Cell Attachment with Corruption of the Viral Fusion Machinery. J Biol Chem. 2011; 286(44):37945-54. Epub 2011/07/30. doi: 10.1074/jbc.M111.256248. PubMed PMID: 21799008; PubMed Central PMCID: PMC3207398.
39. Marcink TC, Yariv E, Rybkina K, Mas V, Bovier FT, des Georges A, et al. Hijacking the Fusion Complex of Human Parainfluenza Virus as an Antiviral Strategy. mBio. 2020; 11(1). Epub 2020/02/13. doi: 10.1128/mBio.03203-19. PubMed PMID: 32047132; PubMed Central PMCID: PMC7018645.
40. Bottom-Tanzer SF, Rybkina K, Bell JN, Alabi CA, Mathieu C, Lu M, et al. Inhibiting Human Parainfluenza Virus Infection by Preactivating the Cell Entry Mechanism. MBio. 2019; 10(1). doi: 10.1128/mBio.02900-18. PubMed PMID: 30782664; PubMed Central PMCID: PMC6381285.
41. Greengard O, Poltoratskaia N, Leikina E, Zimmerberg J, Moscona A. The anti-influenza virus agent 4-GU-DANA (Zanamivir) inhibits cell fusion mediated by human parainfluenza virus and influenza virus HA. J Virol. 2000; 74(23):11108-14. doi: 10.1128/jvi.74.23.11108-11114.2000. PubMed Central PMCID: PMC113191.
42. Rodrigues Toste de Carvalho AL, Liu HY, Chen YW, Porotto M, Moscona A, Snoeck HW. The in vitro multilineage differentiation and maturation of lung and airway cells from human pluripotent stem cell-derived lung progenitors in 3D. Nat Protoc. 2021; 16(4):1802-29. Epub 2021/03/03. doi: 10.1038/s41596-020-00476-z. PubMed PMID: 33649566.
43. Molloy SS, Bresnahan PA, Leppla SH, Klimpel KR, Thomas G. Human furin is a calcium-dependent serine endoprotease that recognizes the sequence Arg-X-X-Arg and efficiently cleaves anthrax toxin protective antigen. J Biol Chem. 1992; 267(23):16396-402. Epub 1992/08/15. PubMed PMID: 1644824.
44. Sakaguchi T, Fujii Y, Kiyotani K, Yoshida T. Correlation of proteolytic cleavage of F protein precursors in paramyxoviruses with expression of the fur, PACE4 and PC6 genes in mammalian cells. J Gen Virol. 1994; 75 (Pt 10):2821-7. Epub 1994/10/01. doi: 10.1099/0022-1317-75-10-2821. PubMed PMID: 7931173.
45. Harbig A, Mernberger M, Bittel L, Pleschka S, Schughart K, Steinmetzer T, et al. Transcriptome profiling and protease inhibition experiments identify proteases that activate H3N2 influenza A and influenza B viruses in murine airways. J Biol Chem. 2020; 295(33):11388-407. Epub 2020/04/19. doi: 10.1074/jbc.RA120.012635. PubMed PMID: 32303635; PubMed Central PMCID: PMCPMC7450141.
46. Sanchez-Guzman D, Boland S, Brookes O, Mc Cord C, Lai Kuen R, Sirri V, et al. Long-term evolution of the epithelial cell secretome in preclinical 3D models of the human bronchial epithelium. Sci Rep. 2021; 11(1):6621. Epub 2021/03/25. doi: 10.1038/s41598-021-86037-0. PubMed PMID: 33758289; PubMed Central PMCID: PMCPMC7988136.
47. Rijsbergen LC, Schmitz KS, Begeman L, Drew-Bear J, Gommers L, Lamers MM, et al. Modeling Infection and Tropism of Human Parainfluenza Virus Type 3 in Ferrets. mBio. 2022:e0383121. Epub 2022/02/16. doi: 10.1128/mbio.03831-21. PubMed PMID: 35164568; PubMed Central PMCID: PMCPMC8844927.
48. Green MG, Huey D, Niewiesk S. The cotton rat (Sigmodon hispidus) as an animal model for respiratory tract infections with human pathogens. Lab Anim (NY). 2013; 42(5):170-6. Epub 2013/04/23. doi: 10.1038/laban.188. PubMed PMID: 23604159.

We claim:

1. A composition useful for treating or preventing HPIV3, the composition comprising at least one component effective for blocking HPIV3 F cleavage and/or viral spread.

2. The composition of claim 1, wherein the at least one component inhibits TMPRSS2 expression, thereby blocking cleavage of F0.

3. The composition of claim 2, wherein the at least one component includes at least one serine protease inhibitor.

4. The composition of claim 3, wherein the at least one serine protease inhibitor includes at least one of the following: alirocumab, alpha-1-proteinase inhibitor, apixaban, aprotinin, argatroban, benzamidine, berotralstat, betrixaban, bivalirudin, camostat, CGS-27023, cholesterol sulfate, chymostatin, conestat alfa, dabigatran, dabigatran etexilate, darexaban, desirudin, edoxaban, evolocumab, fondaparinux, gabexate, GW-813893, human C1-esterase inhibitor, idraparinux, lepirudin, letaxaban, leupeptin, melagatran, nafamostat, otamixaban, rivaroxaban, rosmarinic acid, sivelestat, ulinastatin, and ximelagatran.

5. The composition of claim 4, wherein the at least one serine protease inhibitor includes at least one of the following: aprotinin, camostat, leupeptin, and nafamostat.

6. The composition of claim 5, wherein the at least one serine protease inhibitor includes nafamostat.

7. The composition of claim 5, wherein the at least one serine protease inhibitor includes at least one of aprotinin and leupeptin.

8. The composition of claim 6, wherein the at least one serine protease inhibitor includes at least one of aprotinin and leupeptin.

9. Use of the composition claim 3 in preparing a pharmaceutical composition for treating or preventing a HPIV3 infection.

10. A pharmaceutical composition for treating or preventing a HPIV3 infection, the pharmaceutical composition comprising the composition of claim 3.

11. A pharmaceutical composition for treating or preventing a HPIV3 infection, the pharmaceutical composition comprising the composition of claim 4.

12. A method of treating or preventing a HPIV3 infection by administering the composition of claim 3.

13. A method of treating or preventing a HPIV3 infection by administering the composition of claim 4.

14. A method of treating or preventing a HPIV3 infection by administering the pharmaceutical composition of claim 10.

15. A method of treating or preventing a HPIV3 infection by administering the pharmaceutical composition of claim 11.
